(19) United States
(12) Patent Application Publication
(10) Pub. No.: US 2022/0241141 A1
(43) Pub. Date: Aug. 4, 2022

(54) MULTIFUNCTION KNEE BRACE OR SLEEVE

(71) Applicant: G Force Braces, LLC, Manchester, NH (US)

(72) Inventors: Michael N. Bordieri, JR., Katonah, NY (US); Danielle N. Calvello, Katonah, NY (US); Douglas M. Goumas, BEDFORD, NH (US)

(21) Appl. No.: 17/649,720

(22) Filed: Feb. 2, 2022

Related U.S. Application Data
(60) Provisional application No. 63/199,941, filed on Feb. 4, 2021.

(51) Int. Cl. A61H 9/00; A61F 7/02; A61F 5/01

(52) U.S. Cl. CPC A61H 9/0092; A61H 9/0007; A61F 7/02; A61F 2007/0054; A61H 2209/00; A61H 2201/5038; A61H 2201/5082; A61F 5/0109

(57) ABSTRACT

A multifunction sleeve for a jointed limb is provided. The sleeve may include multiple sections each with one or more inflatable chambers. One or more temperature sensors, one or more range of motion sensors, one or more thermal elements are provided for application of heat or cold therapy. A control device is provided which is adapted to: receive temperature data from the temperature sensors; receive range of motion data from the range of motion sensors; receive temperature data from the thermal elements; control timing, order and duration of inflation and deflation of the inflatable chambers; and control application, timing, temperature, and duration of the heat or cold therapy. A user device may be adapted to receive, store and analyze the temperature and range of motion data received from the control device and/or the sleeve. Potential adverse events may be predicted based on the data received.

This application claims the benefit of U.S. provisional patent application No. 63/199,941 filed on Feb. 4, 2021, which is incorporated herein and made a part hereof by reference.

### BACKGROUND OF THE INVENTION

The present invention relates to the field of medical braces and sleeves. More specifically, the present invention relates to a medical brace or sleeve for the lower extremity that combines various modalities for improved patient treatment and recovery, including edema/lymphedema control, deep vein thrombosis (DVT) prophylaxis, skin temperature sensors, cold and thermal therapy, and range of motion monitoring.

Prior art braces, sleeves, and wraps are known that provide edema/lymphedema control and deep vein thrombosis (DVT) prophylaxis. In addition, braces, sleeves, and wraps having temperature sensors and range of motion sensors are also known. Cold and thermal wraps are also known in the art.

However, it would be advantageous to provide a device that combines all five of these modalities to synergistically improve patient treatment and recovery outcomes, especially with regard to knee surgery. While the device disclosed herein is described as being applied to a knee joint and lower leg of a patient, it should be appreciated by those skilled in the art that the same concepts can be adapted to treat other joints.

The methods, systems, and apparatus of the present invention provide the foregoing and other advantages.

### SUMMARY OF THE INVENTION

The present invention relates to a medical brace or sleeve for the lower extremity that combines various modalities for improved patient treatment and recovery.

In accordance with one example embodiment of the present invention, a multifunction sleeve for a jointed limb is provided. The sleeve comprises a first section comprising a first set of inflatable chambers, a second section proximal of the first section comprising a second set of inflatable chambers, and a third section proximal of the second section comprising at least one additional inflatable chamber. A control device may be provided for controlling inflation and deflation of the inflatable chambers. The first set of inflatable chambers, the second set of inflatable chambers, and the at least one additional inflatable chamber may be inflated in sequence from the most distal to the most proximal to reduce swelling in a joint and/or limb of a patient. Then, the first set of inflatable chambers, the second set of inflatable chambers, and the at least one additional inflatable chamber may be deflated in sequence from the most distal to the most proximal.

The control device may cycle the inflation and the deflation of the first set of inflatable chambers, the second set of inflatable chambers, and the at least one additional inflatable chamber such that after inflation in the sequence the chambers are deflated in the same sequence from the most distal to the most proximal. The control device may repeatedly cycle the inflation and the deflation of the chambers for a programmable period of time. A programmable wait period may be provided between the cycles.

In addition, the control device may be adapted to control the inflation and the deflation of the first set of inflatable chambers, the second set of inflatable chambers, and the at least one additional inflatable chamber independently of one another.

The second section of the sleeve may further comprise one or more temperature sensors. The control device may receive temperature readings from the one or more temperature sensors.

The third section of the sleeve may further comprise at least one of one or more range of motion sensors and a thermal element for application of heat or cold therapy. The control device may receive range of motion data from the one or more range of motion sensors and temperature data from the thermal element.

Additionally or alternately, the sleeve may further comprise one or more thermal elements for the application of heat or cold to at least one of the first section, the second section, and the third section. The one or more thermal elements may each comprise one of a gel pack, a conduit for thermal fluid integrated into the sleeve, and a thermal wrap affixed to or integral with the sleeve.

In one example embodiment of the present invention, one or more thermal elements may be provided which comprise at least one conduit section. A thermal unit may be provided in communication with the control device for providing heated or cooled fluid to the at least one conduit section. The thermal unit may be one of wired or wirelessly connected to the control device.

In a further example embodiment of the present invention, the first set of inflatable chambers may comprise three inflatable chambers. The second set of inflatable chambers may also comprise three inflatable chambers.

In another example embodiment of the present invention, the sleeve may further comprise a frame portion extending at least in the third section and comprising an upper portion and a lower portion. A hinge portion may be provided for movably connecting the upper portion and the lower portion which allows for bending of the frame portion about a knee of a patient. The frame portion may be removably fixed to at least the third section of the sleeve. One or more range of motion sensors may be fixed to the rigid frame portion.

The first set of inflatable chambers, the second set of inflatable chambers, and the at least one additional inflatable chamber may each be inflated and maintained at a constant pressure to provide a rigid sleeve. Also, at least one of the first set of inflatable chambers, the second set of inflatable chambers, and the at least one additional inflatable chamber may be inflated and maintained at a constant pressure to provide rigid portions of the sleeve.

In one example embodiment, the sleeve may further comprise one or more temperature sensors, one or more range of motion sensors, and one or more thermal elements for the application of heat or cold therapy. The control device may be adapted to at least one of: receive temperature data from the one or more temperature sensors; receive range of motion data from the one or more range of motion sensors; receive temperature data from the one or more thermal elements; control timing, order and duration of the inflation and the deflation of the first set of inflatable chambers, the second set of inflatable chambers, and the at least one additional inflatable chamber; and/or control application, timing, temperature, and duration of the heat or cold therapy.

A communication module enabling communication between the sleeve and the control device may be provided. A microprocessor may be provided which is in communication with the communication module and with the one or more temperature sensors, the one or more range of motion sensors, the one or more thermal elements, and the inflatable chambers for processing commands from the control device and providing data to the control device via the communication module.

An air pump may be provided which is connected to the sleeve and in communication with the control device for inflating of the first set of inflatable chambers, the second set of inflatable chambers, and the at least one additional inflatable chamber.

A user device may be provided which is in communication with at least one of the control device and the sleeve. The user device may be one of wired or wirelessly connected to the at least one of the control device and the sleeve. The user device may receive, store and analyze data received from the at least one of the control device and the sleeve.

The user device may comprise one of a software application and a web-based software interface. The at least one of the software application and the web-based software interface are adapted to predict an onset of an adverse event based on the data received at the user device.

In one example embodiment of the present invention, the control device may be integral to the user device.

The present invention also encompasses a system for applying multiple modalities to a jointed limb. In one example embodiment of the present invention, the system comprises a flexible sleeve, one or more temperature sensors, one or more range of motion sensors, one or more thermal elements for application of heat or cold therapy, a control device, and a user device in communication with at least one of the control device and the sleeve. The sleeve comprises a first section comprising a first set of inflatable chambers, a second section proximal of the first section comprising a second set of inflatable chambers, and a third section proximal of the second section comprising at least one additional inflatable chamber. The control device may be adapted to: receive temperature data from the one or more temperature sensors; receive range of motion data from the one or more range of motion sensors; receive temperature data from the one or more thermal elements; control timing, order and duration of inflation and deflation of the first set of inflatable chambers, the second set of inflatable chambers, and the at least one additional inflatable chamber; and control application, timing, temperature, and duration of the heat or cold therapy. The user device may be adapted to receive, store and analyze data received from the at least one of the control device and the sleeve.

The onset of an adverse event based on the data received at the user device may be predicted.

The present invention also encompasses a method for applying multiple modalities to a jointed limb. In one example embodiment of the present invention, such a method comprises providing a flexible sleeve comprising a first section comprising a first set of inflatable chambers, a second section proximal of the first section comprising a second set of inflatable chambers, and a third section proximal of the second section comprising at least one additional inflatable chamber. In addition, the sleeve comprises one or more temperature sensors, one or more range of motion sensors, and one or more thermal elements for application of heat or cold therapy. The method further comprises receiving temperature data from the one or more temperature sensors, receiving range of motion data from the one or more range of motion sensors, receiving temperature data from the one or more thermal elements, controlling timing, order and duration of inflation and deflation of the first set of inflatable chambers, the second set of inflatable chambers, and the at least one additional inflatable chamber, and controlling the application, timing, temperature, and duration of the heat or cold therapy. The method may further comprise predicting an onset of an adverse event based on the received data.

The system and method may include additional features, components, and functionality as discussed above in connection with the example embodiments of the multifunctional sleeve.

### BRIEF DESCRIPTION OF THE DRAWINGS

The present invention will hereinafter be described in conjunction with the appended drawing figures, wherein like reference numerals denote like elements, and:

FIG. 1 shows an example embodiment of a multifunction sleeve in accordance with the present invention;

FIG. 2 shows a further example embodiment of a multifunction sleeve in accordance with the present invention;

FIG. 3 shows an example embodiment of a block diagram of system in accordance with the present invention;

FIG. 4 shows a front view of an example embodiment of a multifunction sleeve with a hinged frame portion in accordance with the present invention;

FIG. 5 shows a rear view of the example embodiment of FIG. 4;

FIG. 6 shows a side view of the example embodiment of FIG. 4; and

FIG. 7 shows a perspective view of the example embodiment of FIG. 4.

### DETAILED DESCRIPTION

The ensuing detailed description provides exemplary embodiments only, and is not intended to limit the scope, applicability, or configuration of the invention. Rather, the ensuing detailed description of the exemplary embodiments will provide those skilled in the art with an enabling description for implementing an embodiment of the invention. It should be understood that various changes may be made in the function and arrangement of elements without departing from the spirit and scope of the invention as set forth in the appended claims.

The present invention comprises a brace or sleeve which combines five modalities into a single device to synergistically improve recovery from injury or surgery. The present invention is designed to improve recovery, prevent complications from surgery, and monitor and measure patient progress. The device combines edema/lymphedema control, DVT prophylaxis, skin temperature sensors, cold or thermal therapy, and range of motion sensors.

The device may be made of a synthetic material and comprise a sleeve with multiple inflatable chambers. Once the device is turned on the chambers may inflate so that the chambers become relatively rigid to provide support for the knee and/or lower leg, while still allowing for bending of the knee joint. The chambers may also be inflated in sequence from the most distal to the most proximal chambers to aid in circulation, reduce inflammation, and prevent or treat edema/lymphedema and DVT. The device may cover at least a portion of the foot and span up to the mid quadricep area of the leg.

The device may include a rigid frame portion that may extend at least above and below the knee joint and which may be hinged to allow for bending of the knee joint. The rigid frame may be made of one or more of metal, aluminum, plastic, carbon fiber, a composite material, or the like. Depending on the application or injury, the rigid frame portion may extend further above or below the knee joint area. The synthetic material comprising the multiple inflatable chambers may be connected to the rigid frame section and may be removable therefrom for cleaning. For example, the synthetic material may be in the form of a sleeve with multiple inflatable chambers and the sleeve may comprise straps for connecting to the rigid frame portion. The straps may comprise Velcro straps, or straps with buckles or other known types of connectors or fasteners. The sleeve may alternatively comprise holes or other material sections for portions of the rigid frame portion to pass through. Those skilled in the art will appreciate that there may be other ways to connect the sleeve to the frame.

Embodiments of the multifunction sleeve discussed below may be provided with or without such a rigid frame portion.

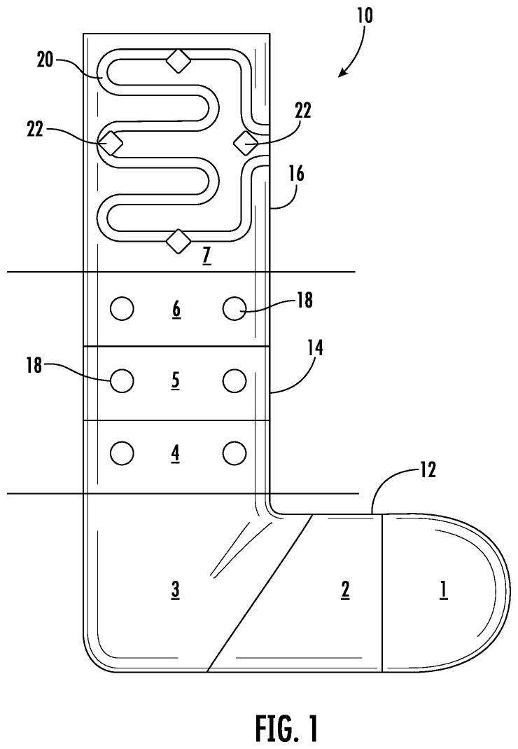

FIG. 1 shows an example embodiment of a multifunction sleeve 10 in accordance with the present invention. The sleeve 10 may be divided into three sections 12, 14, and 16. A first section 12 comprises the lower third (most distal third) of the sleeve 10, which may span from the toes of the patient to just below the calf. This section of the sleeve may comprise a first set of inflatable chambers (e.g., chambers 1, 2, and 3) which can be inflated in sequence from most distal (e.g., chamber 1) to most proximal (e.g., chamber 3) to create pressure on the portions of the foot and lower leg with which they are in contact. The sequential pressurization of the chambers will force the blood up towards the knee and eventually back to the heart. These chambers may be fully inflated and held in the inflated position for a period of time until the cycle is complete, which will prevent blood from returning back into the foot. After the cycle is complete the chambers will deflate, and the process will start again. This sequential inflation process in the lower chambers 1, 2, and 3 will help reduce the amount of swelling that develops in the lower portion of the leg after surgery, helping to prevent edema/lymphedema and DVT. Although the first section 12 is shown with three chambers 1, 2, and 3, it should be appreciated that a lesser or greater number of chambers may be provided.

In the second (middle) section 14 of the sleeve 10, which may span from the calf to approximately four inches below the center of the knee cap, the sleeve 10 will, similarly to the bottom third discussed above, be comprised of a second set of inflatable chambers 4, 5, and 6 which when pressurized in sequence from the most distal (e.g., chamber 4) to the most proximal (e.g., chamber 6) will create pressure on the calf and force the blood up towards the knee and eventually back to the heart. These chambers may be inflated and held in the inflated position for a period of time until the cycle is complete, which will prevent blood from returning back towards the foot. The chambers 4, 5, 6 of the second section 14 may be pressurized in sequence after pressurization of the most proximal chamber (e.g., chamber 3) of the first section 12 is pressurized. The chambers can then be deflated until the process begins again. Although the second section 14 is shown with three chambers 4, 5, and 6, it should be appreciated that a lesser or greater number of chambers may be provided.

The second section 14 of the sleeve 10 may also be equipped with multiple skin temperature sensors 18. These sensors 18 will be strategically placed through this section of the sleeve 10. The purpose of the temperature sensors 18 is to continuously monitor the temperature of the skin below the knee, especially in the calf region. Heat is an early indicator of adverse events such as infection and DVT which this device will be monitoring for.

The third section 16 of the sleeve 10, which is the most proximal section, spans from below the knee to approximately the middle of the quadricep. The third section 16 may comprise at least one additional inflatable chamber 7, which can be inflated (in sequence after the chambers of the first and second sections) and held in the inflated position until the cycle is complete, at which point the chamber can be deflated until the cycle repeats. Alternatively, multiple inflatable chambers may be provided in the third section, which may be pressurized in sequence from most distal to most proximal to force blood from the upper leg towards the heart. The pressurization of the chamber or chambers of the third section 16 will occur in sequence subsequent to the pressurization of the most proximal chamber of the second section 14 (e.g., chamber 6).

The third section 16 may provide compression via the chamber or chambers as well as a cryo/thermal element 20. The cryo or thermal element 20 can be provided by either gel packs (hot or cold) or a continuous flow of hot or cold water through a wrap affixed to or integrated into the sleeve 10, or via conduits provided in the sleeve 10. This third section 16 may also have strategically placed range of motion sensors 22 which can monitor the patient's range of motion. The purpose of the range of motion sensors 22 is to enable clinicians and the patient to monitor the progress of their rehabilitation and return to full function. The posterior (back) side of this section 16 will be a thin, stretchable material which will allow for full extension and flexion of the knee.

The first set of inflatable chambers 1, 2, 3, the second set of inflatable chambers 4, 5, 6, and the at least one additional inflatable chamber 7 may all be inflated in sequence from the most distal to the most proximal to reduce swelling in a joint and/or limb of a patient. Then, the chambers 1-7 may all be deflated in sequence from the most distal to the most proximal.

The chambers 1-7 may all be inflated and kept pressurized so that the entire sleeve 10 becomes rigid to immobilize the lower leg and provide support and protection for the knee after injury or surgery. Alternatively, select chambers can be kept pressurized to provide stability for corresponding portions of the limb.

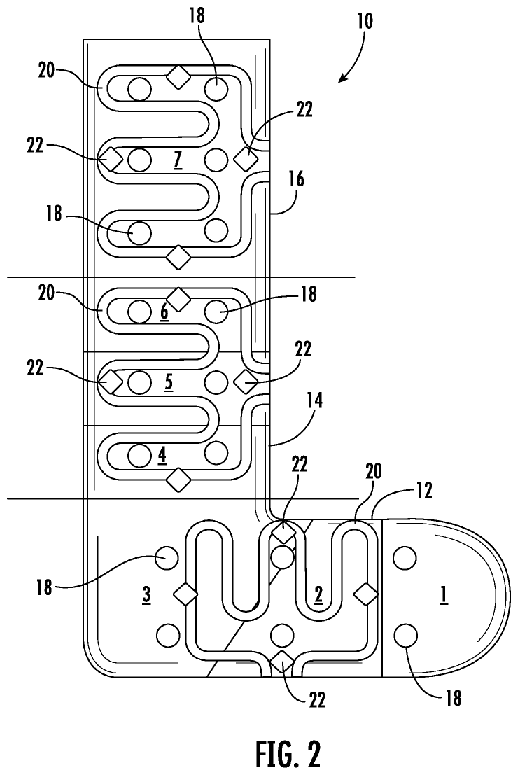

FIG. 2 shows a further example embodiment of the sleeve 10. The cryo or thermal elements 20 may also be provided in one or more of the first section 12 and the second section 14 (e.g., solely in one of the sections, in two of the sections, or in each section) in a similar manner as described above in connection with the third section 16. Temperature sensors 18 may also be provided in the first section 12 and/or the third section 16. Range of motion sensors 22 may also be provided in the second section 14 and/or the third section 16.

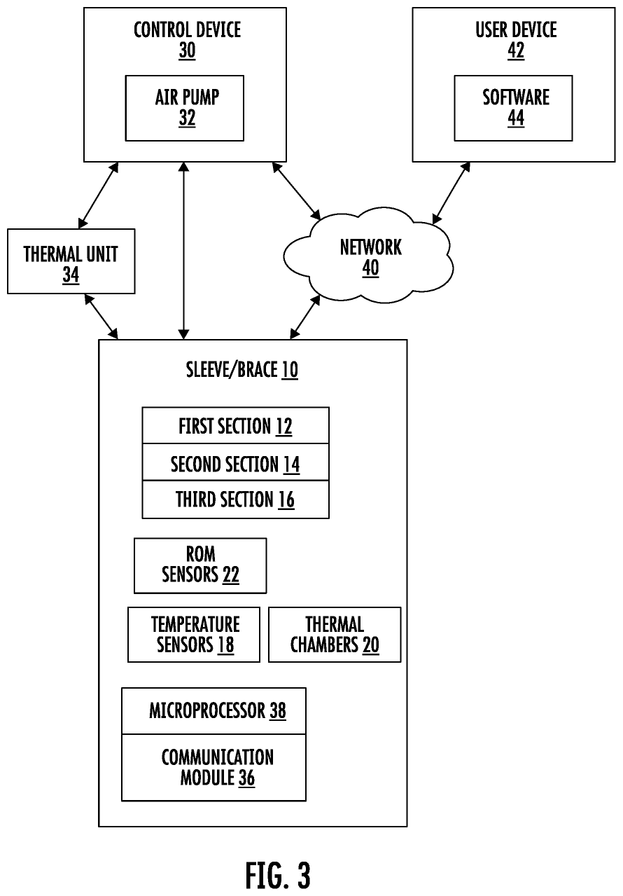

FIG. 3 shows a block diagram of an example embodiment of a system in accordance with the present invention. The system includes sleeve 10 (e.g., as discussed above in connection with FIG. 1), a control device 30, user device 42, and thermal unit 34. The sleeve 10 includes range of motion sensors 22, temperature sensors 18, and thermal elements 20 as discussed above. The control device 30 may be provided for controlling the pressurization sequence of the chambers 1-7 as well as the amount of pressurization. The amount of pressure, the duration of pressurization for each chamber, as well as the timing of the pressurization intervals and duration of the pressurization cycles are programmable and may be configured via the control device 30. The control device 30 may control the inflation and deflation of each chamber 1-7 individually or sequentially.

The control device 30 may also control the application, timing, temperature, and duration of the cryo or thermal therapy, for example by controlling a thermal unit 34 that provides the flow of hot or cold water through, e.g., thermal elements 20 in the sleeve 10. The control device 30 may be connected to the thermal unit 34 in a wired or wireless manner. In an embodiment where a gel pack (hot or cold) is used, the control device 30 may control pressurization of the chamber surrounding the gel pack, including the amount and duration of the pressurization.

The chambers may be pressurized via an air pump 32 integrated into the control device 30 and/or controlled by the control device 30. The air pump 32 may be connected to the sleeve or the chambers 1-7 by air lines. Alternatively, an air pump may be integrated into the sleeve 10, connected to the chambers 1-7, and controlled by the control device 30.

The control device 30 may be connected to the sleeve 10 via a single wired connection or via wired connections to each of the three sections 12, 14, and 16. Alternatively, the sleeve 10 may be fitted with a communication module 36 for enabling wireless communication (e.g., via Bluetooth, Wifi, or LTE data connections) over a network 40 with the control device 30. The sleeve 10 may also be fitted with a microprocessor 38 in communication with the communication module 36 and with the various chambers, sensors, and thermal elements for carrying out the commands from the control device 30 and processing and returning information (e.g., from the inflatable chambers, sensors and thermal elements) to the control device 30 via the communication module 36. For example, the control device 30 may be adapted to receive temperature data from the one or more temperature sensors 18, receive range of motion data from the one or more range of motion sensors 22, and receive temperature data from the one or more thermal elements 20.

The sleeve 10 may also connect to a user device 42 running a software application 44 or a web-based software interface (referred to herein collectively as a software application 44). The user device 42 may comprise a computer, laptop, tablet, smartphone or other Internet based device, which will receive, record, and analyze data from the sleeve 10 and/or from the control device 30. The user device 42 may receive data from the sleeve 10 via a wired connection or wirelessly (e.g., from the microprocessor 38 via the communication module 36 via Bluetooth, Wifi, or LTE data connections over network 40 as discussed above). This data will be stored at the user device 42 or an associated database and used by the patient and clinician to evaluate the progress of patient recovery. This software application 44 will also have the ability to monitor and predict, through a propriety algorithm (and possibly artificial intelligence (AI)) the early onset of an adverse event. These adverse events can be events such a DVT's, infections, limited range of motion, and the like. The purpose of capturing this data is to use it as a tool to create better patient outcomes.

The control device 30 may also be in communication with the user device 42 either via a wired or wireless connection. Alternatively, or in addition to the control device 30, the user device 42 may comprise part or all of the functionality of the control device 30, or the control device 30 may be integrated into the user device 42.

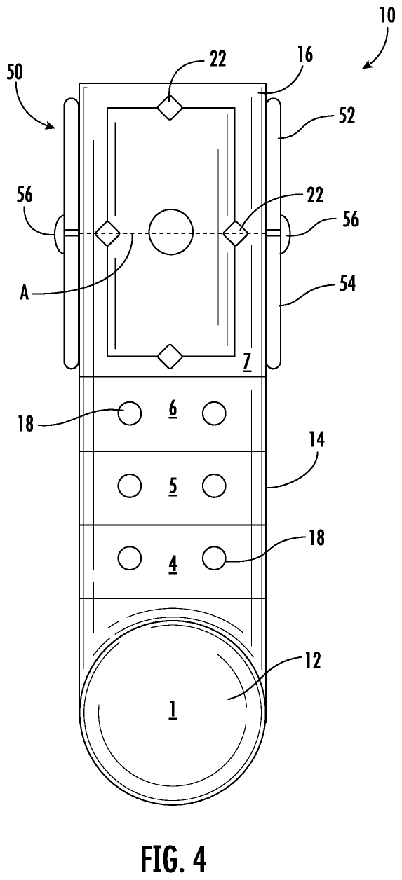

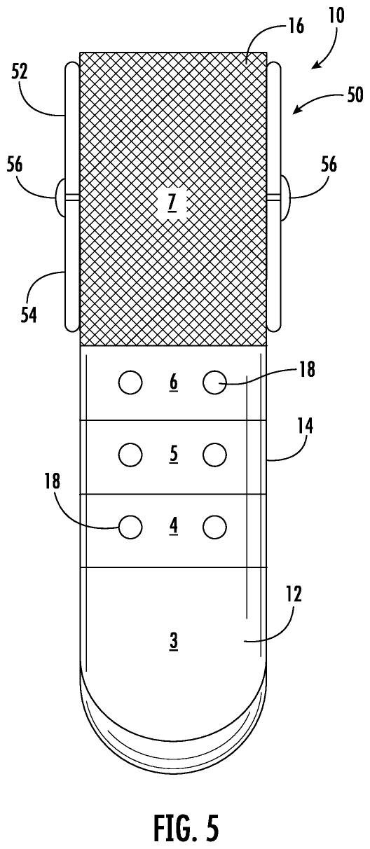

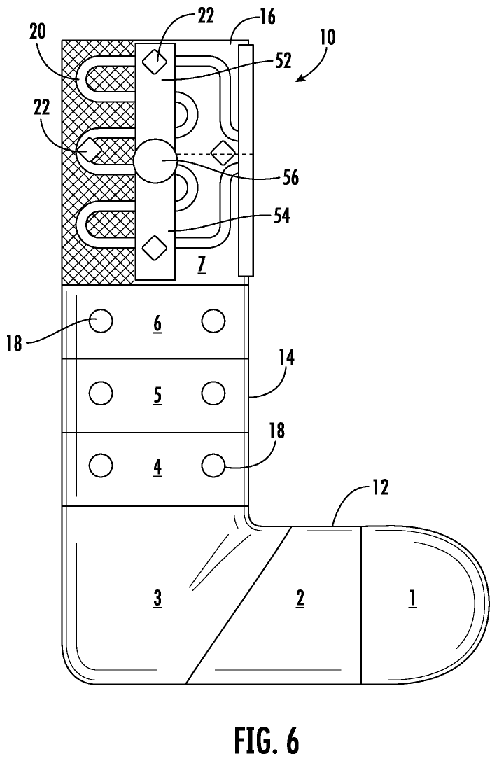

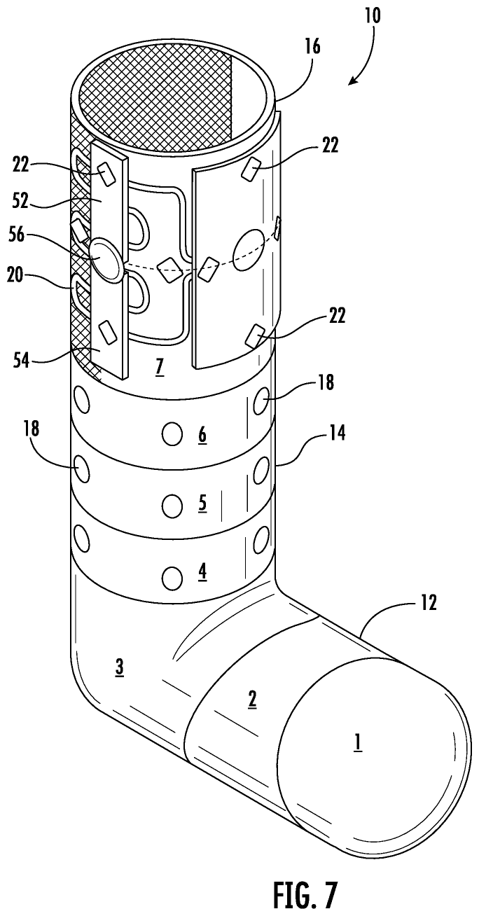

FIGS. 4-7 show views of an example embodiment of the sleeve 10 with a frame portion 50. The frame portion 50 may be rigid and may extend at least in the third section 16, comprising an upper portion 52 positioned above the knee of a patient and a lower portion 54 positioned below the knee of a patient. A hinge portion 56 may be provided for movably connecting the upper portion 52 and the lower portion 54 which allows for bending of the frame portion 50 about a knee of a patient (e.g., about the dashed line A shown in FIG. 4). The frame portion 50 may be removably fixed to at least the third section 16 of the sleeve 10. One or more range of motion sensors 22 may be fixed to the rigid frame portion 50 and/or to the sleeve 17 above and below the knee of the patient. For example, the range of motion sensors 22 may be integrated in or connected to a hinge portion 56 of the frame 50, or be otherwise designed or positioned to sense the motion of the hinge portion 56 or of the movement of the lower portion 54 with respect to the upper portion 52.

The present invention also encompasses a method for applying multiple modalities to a jointed limb. In one example embodiment of the present invention, such a method comprises providing the flexible sleeve 10 as discussed above. The method further comprises receiving temperature data from the one or more temperature sensors 18, receiving range of motion data from the one or more range of motion sensors 22, receiving temperature data from the one or more thermal elements 20, controlling timing, order and duration of inflation and deflation of the first set of inflatable chambers 1, 2, 3 of the first section 12, the second set of inflatable chambers 4, 5, 6 of the second section 14, and the at least one additional inflatable chamber 7 of the third section 16, and controlling the application, timing, temperature, and duration of the heat or cold therapy. The method may further comprise predicting an onset of an adverse event based on the data received at the user device.

The method may include additional features, components, and functionality as discussed above in connection with the example embodiments of the multifunctional sleeve.

It should now be appreciated that the present invention provides advantageous apparatus, systems, and methods for treatment of a joint injury or for recovery from joint surgery.

Although the invention has been described in connection with various illustrated embodiments, numerous modifications and adaptations may be made thereto without departing from the spirit and scope of the invention as set forth in the claims.

What is claimed is:

1. A multifunction sleeve for a jointed limb, comprising:
a first section comprising a first set of inflatable chambers;
a second section proximal of the first section comprising a second set of inflatable chambers; and
a third section proximal of the second section comprising at least one additional inflatable chamber;
wherein a control device is provided for controlling inflation and deflation of the inflatable chambers.

2. A multifunction sleeve in accordance with claim 1, wherein the first set of inflatable chambers, the second set of inflatable chambers, and the at least one additional inflatable chamber are inflated in sequence from the most distal to the most proximal to reduce swelling in at least one of a joint and limb of a patient.

3. A multifunction sleeve in accordance with claim 2, wherein the first set of inflatable chambers, the second set of inflatable chambers and the at least one additional inflatable chamber are deflated in sequence from the most distal to the most proximal.

4. A multifunction sleeve in accordance with claim 3, wherein the control device cycles the inflation and the deflation of the first set of inflatable chambers, the second set of inflatable chambers, and the at least one additional inflatable chamber such that after inflation in the sequence the chambers are deflated in the same sequence from the most distal to the most proximal.

5. A multifunction sleeve in accordance with claim 4, wherein the control device repeatedly cycles the inflation and the deflation of the chambers for a programmable period of time.

6. A multifunction sleeve in accordance with claim 4, wherein a programmable wait period is provided between the cycles.

7. A multifunction sleeve in accordance with claim 1, wherein the control device is adapted to control the inflation and the deflation of the first set of inflatable chambers, the second set of inflatable chambers, and the at least one additional inflatable chamber independently of one another.

8. A multifunction sleeve in accordance with claim 1, wherein the second section further comprises one or more temperature sensors.

9. A multifunction sleeve in accordance with claim 8, wherein the control device receives temperature readings from the one or more temperature sensors.

10. A multifunction sleeve in accordance with claim 1, wherein:
the third section further comprises at least one of one or more range of motion sensors and a thermal element for application of heat or cold therapy; and
the control device receives at least one of range of motion data from the one or more range of motion sensors and temperature data from the thermal element.

11. A multifunction sleeve in accordance with claim 1, further comprising one or more thermal elements for the application of heat or cold to at least one of the first section, the second section, and the third section.

12. A multifunction sleeve in accordance with claim 11, wherein the one or more thermal elements each comprise one of a gel pack, a conduit for thermal fluid integrated into the sleeve, and a thermal wrap affixed to or integral with the sleeve.

13. A multifunction sleeve in accordance with claim 1, further comprising:
one or more thermal elements comprising at least one conduit section;
wherein a thermal unit in communication with the control device provides heated or cooled fluid to the at least one conduit section.

14. A multifunction sleeve in accordance with claim 13, wherein the thermal unit is one of wired or wirelessly connected to the control device.

15. A multifunction sleeve in accordance with claim 1, wherein the first set of inflatable chambers comprises three inflatable chambers.

16. A multifunction sleeve in accordance with claim 1, wherein the second set of inflatable chambers comprises three inflatable chambers.

17. A multifunction sleeve in accordance with claim 1, further comprising a frame portion extending at least in the third section and comprising an upper portion and a lower portion.

18. A multifunction sleeve in accordance with claim 17, further comprising a hinge portion movably connecting the upper portion and the lower portion which allows for bending of the frame portion about a knee of a patient.

19. A multifunction sleeve in accordance with claim 17, wherein the frame portion is removably fixed to at least the third section.

20. A multifunction sleeve in accordance with claim 17, further comprising one or more range of motion sensors fixed to the rigid frame portion.

21. A multifunction sleeve in accordance with claim 1, wherein the first set of inflatable chambers, the second set of inflatable chambers, and the at least one additional inflatable chamber are each inflated and maintained at a constant pressure to provide a rigid sleeve.

22. A multifunction sleeve in accordance with claim 1, wherein at least one of the first set of inflatable chambers, the second set of inflatable chambers, and the at least one additional inflatable chamber is inflated and maintained at a constant pressure to provide rigid portions of the sleeve.

23. A multifunction sleeve in accordance with claim 1, further comprising:
one or more temperature sensors;
one or more range of motion sensors; and
one or more thermal elements for the application of heat or cold therapy.

24. A multifunction sleeve in accordance with claim 23, wherein the control device at least one of:
receives temperature data from the one or more temperature sensors;
receives range of motion data from the one or more range of motion sensors;
receives temperature data from the one or more thermal elements;
controls timing, order and duration of the inflation and the deflation of the first set of inflatable chambers, the second set of inflatable chambers, and the at least one additional inflatable chamber;
controls application, timing, temperature, and duration of the heat or cold therapy.

25. A multifunction sleeve in accordance with claim 24, further comprising:
a communication module enabling communication between the sleeve and the control device;
a microprocessor in communication with the communication module and with the one or more temperature sensors, the one or more range of motion sensors, the one or more thermal elements, and the inflatable chambers for processing commands from the control device and providing data to the control device via the communication module.

26. A multifunction sleeve in accordance with claim 1, wherein an air pump is connected to the sleeve and in communication with the control device for inflating of the first set of inflatable chambers, the second set of inflatable chambers, and the at least one additional inflatable chamber.

27. A multifunction sleeve in accordance with claim 1, wherein a user device is in communication with at least one of the control device and the sleeve.

28. A multifunction sleeve in accordance with claim 27, wherein:
the user device is one of wired or wirelessly connected to the at least one of the control device and the sleeve;
the user device receives, stores and analyzes data received from the at least one of the control device and the sleeve.

29. A multifunction sleeve in accordance with claim 27, wherein the user device comprises at least one of a software application and a web-based software interface.

30. A multifunction sleeve in accordance with claim 29, wherein the at least one of the software application and the web-based software interface are adapted to predict an onset of an adverse event based on the data received at the user device.

31. A multifunction sleeve in accordance with claim 27, wherein the control device is integral to the user device.

32. A system for applying multiple modalities to a jointed limb, comprising:
a flexible sleeve comprising:
a first section comprising a first set of inflatable chambers, a second section proximal of the first section comprising a second set of inflatable chambers, and a third section proximal of the second section comprising at least one additional inflatable chamber; and
one or more temperature sensors, one or more range of motion sensors, and
one or more thermal elements for application of heat or cold therapy;
a control device, adapted to:
receive temperature data from the one or more temperature sensors;
receive range of motion data from the one or more range of motion sensors;
receive temperature data from the one or more thermal elements;
control timing, order and duration of inflation and deflation of the first set of inflatable chambers, the second set of inflatable chambers, and the at least one additional inflatable chamber; and
control application, timing, temperature, and duration of the heat or cold therapy; and
a user device in communication with at least one of the control device and the sleeve, the user device adapted to receive, store and analyze data received from the at least one of the control device and the sleeve.

33. The system in accordance with claim 32, wherein an onset of an adverse event based on the data received at the user device is predicted.

34. A method for applying multiple modalities to a jointed limb, comprising:
providing a flexible sleeve comprising:
a first section comprising a first set of inflatable chambers, a second section proximal of the first section comprising a second set of inflatable chambers, and a third section proximal of the second section comprising at least one additional inflatable chamber; and
one or more temperature sensors, one or more range of motion sensors, and one or more thermal elements for application of heat or cold therapy;
receiving temperature data from the one or more temperature sensors;
receiving range of motion data from the one or more range of motion sensors;
receiving temperature data from the one or more thermal elements;
controlling timing, order and duration of inflation and deflation of the first set of inflatable chambers, the second set of inflatable chambers, and the at least one additional inflatable chamber;
controlling the application, timing, temperature, and duration of the heat or cold therapy.

35. The method in accordance with claim 34, further comprising predicting an onset of an adverse event based on the received data.
